(19) United States
(12) Patent Application Publication
(10) Pub. No.: US 2013/0071847 A1
(43) Pub. Date: Mar. 21, 2013

(54) METHODS AND MATERIALS FOR DETERMINING THE SOURCE OF WASTE

(71) Applicants: Ashley Michelle Burnett, Knoxville, TN (US); Chesleigh Nicole Winfree, Knoxville, TN (US); Kathryn Ellen Oliff, Knoxville, TN (US); Dawn Irion, Knoxville, TN (US)

(72) Inventors: Ashley Michelle Burnett, Knoxville, TN (US); Chesleigh Nicole Winfree, Knoxville, TN (US); Kathryn Ellen Oliff, Knoxville, TN (US); Dawn Irion, Knoxville, TN (US)

(73) Assignee: BioPet Vet Lab, Knoxville, TN (US)

(21) Appl. No.: 13/236,204

(22) Filed: Sep. 19, 2011

(51) Int. Cl. C12Q 1/68; C12M 1/26

(52) U.S. Cl. 435/6.12; 435/309.1

(57) ABSTRACT

A method for identifying the source of animal waste is provided. The method includes taking DNA samples from a known group of animals, conducting DNA analysis on the DNA samples to prepare a genetic profile for each animal from the group, preparing a database of the genetic profiles, collecting a specimen of waste from an unknown source, conducting DNA analysis on the specimen, and comparing the DNA analysis from the specimen to the database to determine the source of the waste.

### BACKGROUND OF THE INVENTION

The present invention relates to methods and materials for determining sources of waste. More specifically, the present invention relates to methods and materials for determining the source of fecal matter.

Many communities deal with the issue of animal waste removal. It is desirable in communities, such as neighborhoods, condominium complexes, and apartment complexes to maintain common areas free of animal waste. Many such communities attempt to monitor such activity and fine owners of animals who do not clean up after their pets, but the communities find such monitoring systems to be time- and cost-prohibitive.

Moreover, the typical waste from a canine contains about three billion bacteria in addition to other pests, all of which can pollute lakes streams, and rivers. For example, canine fecal matter may contain parasites, such as cryptosporidium, giardia, hookworms, roundworms, and tapeworms and bacteria and viruses, such as salmonella, Escherichia coli, campylobacter, and leptospira. These pests, which can survive in the soil are capable of transferring from dog-to-dog and dog-to-human. Additionally, they can lead to fever, kidney disorders, headaches, vomiting, diarrhea, and muscle aches and cramps.

Children are at particular risk of infection in areas where dog waste is allowed to contaminate the soil, because they often play on the ground with their hands and frequently put their hands in their mouths. They also drop toys and pacifiers on the ground and then place them in their mouths. Toxocara canis, a roundworm found in dog waste is particularly dangerous to children and can, in some instances, cause blindness.

Additionally, the Environmental Protection Agency places dog waste in the same health category as oil and toxic chemicals. Over the last several years, E. coli bacteria from dog waste caused a public park in Austin, Tex. to shutdown and bacterial source tracking studies in watersheds in the Seattle, Wash. area found that nearly 20% of the bacteria isolates that could be matched with host animals were matched with dogs. As can be seen, the problem with uncollected dog waste is not limited to annoyance, but is a genuine health and pollution issue that must be dealt with by communities.

### SUMMARY OF THE INVENTION

In one aspect, the present invention is a method for identifying the source of animal waste. The method includes taking DNA samples from a known group of animals, conducting DNA analysis on the DNA samples to prepare a genetic profile for each animal from the group, preparing a database of the genetic profiles, collecting a specimen of waste from an unknown source, conducting DNA analysis on the specimen, and comparing the DNA analysis from the specimen to the database to determine the source of the waste.

In another aspect, the invention is a kit for collecting and analyzing animal waste. The kit includes at least one first DNA sample collector to collect DNA samples from a known group of animals, wherein the DNA samples from the known group of animals can be extracted from cheek cells, saliva, fur, blood, or waste, and at least one second DNA sample collector for collecting a sample of fecal matter from an unknown animal.

These and other aspects of the invention will be understood and become apparent upon review of the specification by those having ordinary skill in the art.

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in or are obvious from the following detailed description. It is to be understood by one of ordinary skill in the art that the present discussion is a description of exemplary embodiments only, and is not intended as limiting the broader aspects of the present invention.

In one aspect, the present invention is a method for identifying the source of animal waste. The method includes taking DNA samples from a known group of animals, conducting DNA analysis on the DNA samples to prepare a genetic profile for each animal from the group, preparing a database of the genetic profiles, collecting a specimen of waste from an unknown source, conducting DNA analysis on the specimen, and comparing the DNA analysis from the specimen to the database to determine the source of the waste.

As used herein, the terms “fecal matter” and “waste” are used interchangeably. Additionally, as used herein, the terms “dog” and “canine” are used interchangeably.

In some embodiments, the method may be implemented in a community such as an apartment or condominium complex or a housing neighborhood. In other embodiments, the method may be implemented in a larger community, such as throughout a town. In yet other embodiments, the method may be implemented in a park where people exercise their pets.

The method is particularly useful for the identification of the source of canine waste.

For ease of reference, the method will be described with respect to the identification of the source of canine fecal matter in an apartment complex. One having ordinary skill in the art will recognize that the method is applicable in other communities, such as, but not limited to, the types of communities listed above. The method shall not, therefore, be limited to apartment communities as discussed herein.

Additionally, and for ease of reference, the invention will be described with reference to canines, but shall not be so limited. It should be appreciated by those having ordinary skill in the art that the invention may be utilized with animals other than dogs. The method shall not, therefore, be limited to dog waste as discussed herein.

In one embodiment, the method may be conducted by taking a DNA sample from pets living in a particular area, for example, an apartment complex. It may be desirable to take a DNA sample from all animals living in the apartment complex. In some embodiments, it may be desirable to take a DNA sample from the dogs living in the apartment complex. The DNA samples may be taken from one or more of the cheek cells, saliva, fur, blood, or fecal matter from the dogs.

After the DNA samples are taken, the samples may be analyzed to develop a genetic profile from each dog. The genetic profile may be determined by one or more of hybridization, Polymerase Chain Reaction, size fractionation, DNA sequencing, DNA microarrays, high density fiber-optic arrays of beads, primer extension, mass spectrometry, and whole-genome sampling, as well as other methods known in the art. This genetic profile may then be stored in a database, such as a computer database in or on a computer-readable medium. In other embodiments, the genetic profile may be printed and stored in a physical file. In all embodiments, the collected genetic profiles should be stored in a manner that will facilitate later searching of the profiles to enable comparison of the genetic profiles to a genetic profile generated from an unknown sample of waste.

The method further includes taking a sample of waste material when waste from an unknown animal is located in the apartment complex. The sample from the unknown animal may then be subjected to DNA analysis to develop a genetic profile of the unknown animal. The genetic profile may be developed using methods of DNA analysis known in the art. Exemplary methods of DNA analysis include, but are not limited to hybridization, Polymerase Chain Reaction, size fractionation, DNA sequencing, DNA microarrays, high density fiber-optic arrays of beads, primer extension, mass spectrometry, and whole-genome sampling, as well as other methods known in the art. The genetic profile of the unknown animal may then be compared to the genetic profiles in the database to determine whether the waste originated from an animal in the apartment complex.

In some embodiments, it may be desirable to stabilize the sample taken from the unknown waste. One of the difficulties in DNA profiling is the RNA and DNA degradation during collection, storage, and transportation of samples. By utilizing a stabilizer in biological samples, the changes in the gene-expression patterns that occur due to nonspecific DNA and RNA degradation can be avoided, resulting in more accurate DNA analysis of the sample. Various stabilizers are available, including, but not limited to, RNASafer° by Omega bio-tek, RNAlater™ by Qiagen, and Xpedition Lysis/Stabilization Solution by Zymo Research.

When a DNA stabilizer is used, it may be desirable to ensure the surface of the sample is completely covered by the stabilizer. In other embodiments, it may be desirable to mix the sample in the stabilizer such that the stabilizer penetrates the sample.

After the sample is obtained, the DNA may be extracted from the fecal matter. Various methods of DNA extraction are known in the art and may be utilized in conjunction with the present method. A general description of DNA extraction techniques follows, but any DNA extraction technique known in the art may be utilized in conjunction with the present invention.

In a general method, DNA extraction may be conducted by first lysing the cells (breaking the cells open), to expose the DNA within the cells. This step may be conducted by grinding or sonicating the sample. Vortexing with phenol (sometimes heated) is often effective for breaking down proteinacious cellular walls or viral capsids. After the cells are opened, the membrane lipids may be removed, for example, by adding a surfactant or detergent to the sample. Optionally, DNA associated proteins, as well as other cellular proteins may be degraded with the addition of a protease. Precipitation of the protein is aided by the addition of a salt such as ammonium or sodium acetate. When the sample is vortexed with phenol-chloroform and centrifuged, the proteins will remain in the organic phase and can be drawn off carefully. The DNA will be found at the interface between the two phases. DNA is then precipitated by mixing with cold ethanol or isopropanol and then centrifuging. The DNA is insoluble in the alcohol and will come out of solution, and the alcohol serves as a wash to remove the salt previously added. The resultant DNA pellet may then be washed with cold alcohol again and centrifuged for retrieval of the pellet. If desired, the DNA can be re-suspended in a buffer such as Tris or TE.

It may be desirable to utilize a commercially available kit for DNA extraction. Some commercially available DNA extraction kits include, but are not limited to, ZR Fecal DNA MiniPrep™ from Zymo Research, QIAamp™ DNA mini kit from Qiagen, ExtractMaster Fecal DNA Extraction Kit from Epientre, and UltraClean Fecal DNA Isolation Kit by MoBio Laboratories.

Additionally, it may be desirable to modify the extraction method by utilizing additional lysis solution to the fecal sample to sufficiently extract the DNA from the cells. For example, when the waste sample has been stabilized such that it is in solution and the extraction is conducted utilizing the ZR Fecal DNA MiniPrep™ system, which is designed for extraction from solids, it may be desirable to add additional lysis solution to efficiently conduct the extraction.

Once the DNA is extracted from the waste sample, the extracted DNA may be analyzed to develop a genetic profile of the extracted DNA, as discussed above.

Additionally, as discussed above, the genetic profile of the DNA extracted from the unknown waste sample may be compared to the genetic profiles in the database on the computer-readable medium to determine the source of the unknown waste sample. Any algorithms useful for multi-locus genotype analysis may be used in the methods of the invention, for example classic assignment algorithms. Suitable algorithms include those described in Rannala & Mountain (1997) Proc. Natl. Acad. Sci. U.S.A. 94:9197-9201 and Cornuet et al. (1999) Genetics 153: 1989-2000 and variations thereof.

As used herein, “computer-readable medium” refers to any available medium that can be accessed by computer and includes both volatile and nonvolatile media, removable and non-removable media. By way of example, and not limitation, computer readable media may comprise computer storage media and communication media. Computer storage media includes both volatile and nonvolatile, removable, and non-removable media implemented in any method or technology for storage of information, such as computer-readable instructions, data structures, program modules, or other data. Computer storage media include, but are not limited to RAM, FOM, EEPROM, flash memory or other memory technology, CD-ROM, DVD or other optical disk storage, magnetic cassettes, magnetic tapes, magnetic disk storage or other magnetic storage devices, or any other computer storage media. Communication media typically embody computer-readable instructions, data structures, program modules, or other data in a modulated data signal, such as a carrier wave or other transport mechanism that includes any information delivery media. The term “modulated data signal” means a signal that has one or more of its characteristics set or changed in such a manner as to encode information in the signal. By way of example, and not limitation, communication media include wired media, such as a wired network or direct-wired connection, and wireless media, such as acoustic, RF infrared, and other wireless media. A combination of any of the above should also be included within the scope of computer-readable media.

In some embodiments, the computer readable medium comprises a substrate having stored thereon a database of genetic profiles of animals that have been collected as part of the presently-contemplated method. The genetic profile obtained from the unknown waste sample may then be entered into or onto the computer readable medium and the unknown genetic profile may be compared, either by an algorithm or manually, to the genetic profiles stored in the database to determine the source of the waste sample.

In another aspect, the invention is a computer readable medium comprising stored thereon a database having stored thereon genetic profiles developed from DNA analysis of a set of known animals and computer-executable instructions for implementing a method for comparing a genetic profile from an unknown animal with the genetic profiles stored on the database for determining the source of the unknown genetic profile.

In another aspect, the invention is a kit for collecting and analyzing animal waste. The kit includes at least one first DNA sample collector to collect DNA samples from a known group of animals, wherein the DNA samples from the known group of animals can be extracted from cheek cells, saliva, fur, blood, or waste, and at least one second DNA sample collector for collecting a sample of fecal matter from an unknown animal.

In one embodiment, the first DNA sample collector is designed for collecting a sample of cheek cells from an animal, for example, a dog. The sample collector includes a buccal swab and a vessel for storing the buccal swab. It may be desirable for the vessel to include a stabilizer, such as those discussed above. In another embodiment, the first DNA sample collector may include a vessel for containing a hair sample from an animal, for example, a dog. In yet another embodiment, the first DNA sample collector may include a syringe and needle for collecting a blood sample from an animal, for example a dog, and a vessel for storing the blood sample. In some embodiments, it may be desirable for the vessel to include a stabilizer, such as those discussed above. In a different embodiment, the first DNA sample collector may include a device for collecting a waste sample, such as a scoop or tongs, and a vessel for storing the waste sample. In some embodiments, it may be desirable for the vessel to include a stabilizer. Additionally, in all embodiments where it may be desirable to utilize a stabilizer, the stabilizer may be provided separately from the storage vessel and may be added to the storage vessel and when the DNA sample is collected.

It may be desirable to include a plurality of the first DNA sample collector in the kit to facilitate collection of DNA samples from more than one animal, thereby enabling creation of the database of genetic profiles discussed above.

The second DNA sample collector may include a scoop or other instrument for collecting a sample of fecal material. Additionally, the second DNA sample collector may include a vessel for storing the fecal material until the fecal matter can be analyzed to develop a genetic profile. It may be desirable to include a DNA stabilizer as part of the second DNA sample collector, either in the vessel or in a separate container, such that the stabilizer can be added to the vessel upon collection of the fecal matter. The DNA stabilizer may be useful for stabilizing the DNA in the fecal matter until the sample may be subjected to DNA analysis.

In some embodiments, it may be desirable to include a plurality of the second DNA sample collector in the kit to facilitate collection of more than one unknown sample of fecal matter.

The following examples describe exemplary embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### Example 1

This example describes a representative method for identifying the specific source of animal waste, for example canine waste.

A community including 57 dogs participated in a study utilizing the present methods. A buccal swab was collected from each of the 57 dogs and subjected to DNA analysis utilizing known methods to produce a genetic profile for each dog. The genetic profiles are depicted in Table 1.

When unknown fecal matter was located in the community, a sample of the fecal matter was taken, assigned trial number TD0002113, and subjected to DNA analysis according to the following protocol:
1 mL of sample was transferred to a 1.7 mL microcentrifuge tube and centrifuged 1 minute at 7,000 rpm.
Leaving approximately 100 μL, the supernatant was removed and discarded.
The fecal pellet was resuspended with 750 μL of Zymo Lysis Solution.
The suspension was transferred to the ZR BashingBead Lysis Tube.
The tube was secured to the vortex and processed at maximum speed for 5 minutes.
The tube was spun at 10,000×g for 1 minute.
400 μL of supernatant was transferred to a Zymo-Spin IV Spin Filter in a collection tube and spun at 7,000×g for 1 minute.
1,200 μL of Fecal DNA Binding Buffer containing 0.5% beta-mercaptoethanol was added to the collection tube contain the filtrate.
800 μL of the filtrate-binding buffer mixture was transferred to a Zymo-Spin IIC Column in a collection tube and spun at 10,000×g for 1 minute.
The flow was discarded from the collection tube.
The remaining 800 μL of the filtrate-binding buffer mixture was transferred to the same Zymo-Spin IIC Column and spun at 10,000×g for 1 minute.
The collection tube was discarded and replaced with a new tube.
200 μL of DNA Pre-Wash Buffer was applied to the Zymo-Spin IIC Column and spun for 1 minute at 10,000×g.
500 μL of Fecal DNA Wash Buffer was applied to the Zymo-Spin IIC Column and spun for 1 minute at 10,000×g.
The collection tube was discarded and the Zymo-Spin IICColumn was transferred to a clean 1.7 mL microcentrifuge tube.
100 μL of DNA Elution Buffer was applied directly to the column matrix and allowed to incubate 1 minute at room temperature.
The tube was spun at 10,000×g for 30 seconds to elute the DNA.
Zymo-Spin IV-HRC Spin Filter was placed into a clean 1.7 mL microcentrifuge tube. The eluted DNA was applied to the matrix and spun for 1 minute at 8,000×g making the DNA now suitable for PCR.

Using an ABI Verti 96 Well Fast Thermal Cycler, the DNA from sample TD0002113 was amplified in a PCR reaction using the molecular markers Amelogenin, FH2010, FH2054, FH2079, FH2361, Pez01, Pez03, Pez05, Pez06, Pez08, Pez11, Pez12, Pez16, Pez17, Pez20, and Pez21. HiDi Formamide (25 mL) was mixed with 150 uL of MRK500. 2 uL of the PCR product was applied to a 96 well plate containing 10 uL of HiDi Formamide/MRK500. The PCR product was denatured at 95° C. for 5 minutes. The denatured plate was placed on an ABI 3730 DNA Analyzer to extract raw molecular marker data. The raw data was then transferred into BioPet's ABI GeneMapper software where manual analysis of the data was performed. The analysis provided TD0002113 with the genotype shown in Table 2.

TABLE 1
Genetic Profiles of Dogs in Community (Part I)

| | Amel | Amel | FH2010 | FH2010 | FH2054 | FH2054 | FH2079 | FH2079 | FH2361 | FH2361 | PEZ01 | PEZ01 | PEZ03 | PEZ03 | PEZ05 |
|---|---|---|---|---|---|---|---|---|---|---|---|---|---|---|---|
| DN1 | 218 | 218 | 228 | 236 | 153 | 169 | 271 | 271 | 347 | 351 | 118 | 122 | 108 | 124 | 102 |
| DN2 | 182 | 218 | 228 | 236 | 149 | 149 | 271 | 271 | 339 | 347 | 118 | 118 | 124 | 128 | 102 |
| DN3 | 218 | 218 | 232 | 236 | 153 | 157 | 271 | 271 | 351 | 367 | 106 | 114 | 128 | 128 | 110 |
| DN4 | 218 | 218 | 228 | 236 | 153 | 165 | 267 | 271 | 347 | 355 | 114 | 118 | 128 | 128 | 114 |
| DN5 | 218 | 218 | 224 | 228 | 153 | 177 | 267 | 267 | 351 | 355 | 118 | 122 | 122 | 130 | 110 |
| DN6 | 218 | 218 | 232 | 236 | 153 | 157 | 271 | 275 | 343 | 361 | 122 | 130 | 116 | 130 | 102 |
| DN7 | 218 | 218 | 228 | 236 | 149 | 153 | 267 | 267 | 347 | 355 | 118 | 122 | 118 | 122 | 106 |
| DN8 | 0 | 0 | 228 | 228 | 143 | 153 | 271 | 271 | 343 | 347 | 0 | 0 | 122 | 130 | 106 |
| DN9 | 218 | 218 | 224 | 232 | 153 | 165 | 267 | 275 | 347 | 355 | 118 | 126 | 124 | 130 | 94 |
| DN10 | 218 | 218 | 236 | 236 | 149 | 149 | 271 | 275 | 351 | 355 | 110 | 122 | 124 | 130 | 102 |
| DN11 | 182 | 218 | 228 | 228 | 149 | 165 | 271 | 271 | 347 | 347 | 110 | 126 | 130 | 136 | 110 |
| DN12 | 218 | 218 | 228 | 228 | 153 | 153 | 267 | 267 | 343 | 347 | 110 | 122 | 118 | 134 | 110 |
| DN13 | 218 | 218 | 236 | 236 | 153 | 169 | 271 | 275 | 351 | 367 | 118 | 122 | 108 | 122 | 102 |
| DN14 | 182 | 218 | 228 | 232 | 153 | 161 | 271 | 271 | 351 | 351 | 114 | 114 | 124 | 142 | 102 |
| DN15 | 182 | 218 | 232 | 232 | 153 | 165 | 275 | 275 | 351 | 359 | 126 | 130 | 112 | 136 | 102 |
| DN16 | 218 | 218 | 236 | 236 | 153 | 177 | 271 | 275 | 339 | 355 | 118 | 122 | 108 | 128 | 110 |
| DN17 | 182 | 218 | 228 | 228 | 153 | 161 | 271 | 275 | 343 | 359 | 118 | 126 | 124 | 140 | 106 |
| DN18 | 182 | 218 | 224 | 236 | 153 | 169 | 267 | 271 | 351 | 363 | 118 | 118 | 124 | 134 | 102 |
| DN19 | 182 | 218 | 224 | 224 | 153 | 169 | 287 | 287 | 355 | 359 | 122 | 130 | 134 | 142 | 102 |
| DN20 | 218 | 218 | 228 | 236 | 145 | 161 | 271 | 275 | 339 | 349 | 118 | 118 | 124 | 124 | 110 |
| DN21 | 182 | 218 | 232 | 232 | 153 | 165 | 271 | 271 | 339 | 351 | 122 | 122 | 122 | 124 | 106 |
| DN22 | 218 | 218 | 232 | 236 | 161 | 177 | 275 | 275 | 343 | 347 | 114 | 122 | 130 | 136 | 106 |
| DN23 | 182 | 218 | 224 | 236 | 161 | 165 | 271 | 271 | 347 | 351 | 118 | 122 | 128 | 134 | 102 |
| DN24 | 218 | 218 | 224 | 236 | 165 | 165 | 271 | 271 | 355 | 425 | 122 | 122 | 130 | 134 | 102 |
| DN25 | 218 | 218 | 228 | 236 | 153 | 157 | 271 | 275 | 355 | 359 | 126 | 126 | 136 | 142 | 110 |
| DN26 | 182 | 218 | 224 | 228 | 153 | 161 | 271 | 275 | 355 | 359 | 122 | 122 | 130 | 142 | 102 |
| DN27 | 182 | 218 | 228 | 236 | 149 | 161 | 267 | 287 | 347 | 365 | 126 | 126 | 118 | 118 | 102 |
| DN28 | 218 | 218 | 240 | 240 | 149 | 149 | 0 | 0 | 347 | 357 | 0 | 0 | 134 | 146 | 106 |
| DN29 | 0 | 0 | 232 | 236 | 149 | 153 | 267 | 271 | 347 | 355 | 110 | 126 | 118 | 124 | 102 |
| DN30 | 218 | 218 | 232 | 236 | 149 | 165 | 275 | 275 | 339 | 351 | 118 | 122 | 122 | 124 | 110 |
| DN31 | 218 | 218 | 224 | 228 | 169 | 173 | 271 | 271 | 351 | 351 | 118 | 118 | 124 | 130 | 102 |
| DN32 | 182 | 218 | 228 | 236 | 153 | 165 | 275 | 287 | 339 | 347 | 118 | 122 | 112 | 128 | 106 |
| DN33 | 182 | 218 | 224 | 228 | 157 | 169 | 271 | 271 | 351 | 351 | 118 | 118 | 124 | 134 | 110 |
| DN34 | 218 | 218 | 224 | 228 | 149 | 153 | 271 | 275 | 343 | 349 | 118 | 122 | 116 | 124 | 102 |
| DN35 | 182 | 218 | 228 | 236 | 153 | 169 | 267 | 275 | 347 | 347 | 122 | 130 | 118 | 122 | 102 |
| DN36 | 218 | 218 | 232 | 236 | 149 | 149 | 267 | 275 | 347 | 359 | 122 | 130 | 116 | 130 | 0 |
| DN37 | 218 | 218 | 228 | 232 | 165 | 169 | 267 | 267 | 345 | 347 | 118 | 122 | 122 | 124 | 102 |
| DN38 | 218 | 218 | 224 | 224 | 153 | 177 | 271 | 287 | 351 | 351 | 118 | 122 | 128 | 130 | 102 |
| DN39 | 182 | 218 | 228 | 228 | 149 | 157 | 275 | 287 | 347 | 351 | 118 | 122 | 118 | 142 | 102 |
| DN40 | 218 | 218 | 236 | 236 | 153 | 169 | 267 | 271 | 335 | 351 | 118 | 126 | 116 | 122 | 102 |
| DN41 | 182 | 218 | 224 | 236 | 165 | 173 | 271 | 279 | 347 | 355 | 118 | 126 | 124 | 124 | 102 |
| DN42 | 218 | 218 | 232 | 236 | 149 | 153 | 267 | 287 | 347 | 355 | 118 | 122 | 122 | 124 | 102 |
| DN43 | 182 | 218 | 228 | 236 | 157 | 165 | 275 | 275 | 359 | 359 | 114 | 118 | 122 | 124 | 110 |
| DN44 | 182 | 218 | 224 | 228 | 161 | 161 | 271 | 271 | 355 | 355 | 118 | 118 | 124 | 128 | 102 |
| DN45 | 218 | 218 | 228 | 232 | 157 | 165 | 267 | 271 | 347 | 347 | 110 | 122 | 128 | 140 | 106 |
| DN46 | 218 | 218 | 228 | 232 | 153 | 153 | 271 | 275 | 351 | 351 | 126 | 126 | 124 | 140 | 102 |
| DN47 | 218 | 218 | 228 | 228 | 157 | 161 | 275 | 287 | 359 | 363 | 122 | 122 | 128 | 130 | 102 |
| DN48 | 0 | 0 | 228 | 236 | 153 | 153 | 271 | 275 | 0 | 0 | 114 | 118 | 118 | 124 | 102 |
| DN49 | 218 | 218 | 224 | 228 | 161 | 165 | 267 | 271 | 351 | 369 | 126 | 126 | 122 | 122 | 102 |
| DN50 | 0 | 0 | 232 | 236 | 145 | 153 | 267 | 271 | 347 | 351 | 118 | 118 | 128 | 140 | 94 |
| DN51 | 218 | 218 | 228 | 236 | 153 | 173 | 271 | 275 | 351 | 353 | 118 | 130 | 122 | 130 | 110 |
| DN52 | 218 | 218 | 228 | 228 | 153 | 165 | 275 | 275 | 347 | 361 | 118 | 126 | 0 | 0 | 110 |
| DN53 | 218 | 218 | 228 | 232 | 173 | 173 | 267 | 267 | 351 | 363 | 126 | 130 | 130 | 130 | 110 |
| DN54 | 182 | 218 | 228 | 228 | 153 | 161 | 271 | 275 | 343 | 351 | 110 | 122 | 128 | 134 | 102 |
| DN55 | 218 | 218 | 228 | 228 | 157 | 169 | 271 | 271 | 355 | 355 | 122 | 122 | 134 | 134 | 106 |
| DN56 | 218 | 218 | 228 | 232 | 145 | 161 | 271 | 271 | 351 | 359 | 118 | 122 | 116 | 118 | 102 |
| DN57 | 182 | 218 | 228 | 228 | 149 | 153 | 275 | 275 | 347 | 351 | 114 | 118 | 128 | 134 | 110 |

Genetic Profiles of Dogs in Community (Part II)

| | PEZ05 | PEZ06 | PEZ06 | PEZ08 | PEZ08 | PEZ11 | PEZ11 | PEZ12 | PEZ12 |
|---|---|---|---|---|---|---|---|---|---|
| DN1 | 110 | 396 | 404 | 236 | 246 | 363 | 379 | 264 | 264 |
| DN2 | 110 | 392 | 392 | 232 | 236 | 375 | 391 | 264 | 272 |
| DN3 | 110 | 388 | 400 | 230 | 246 | 375 | 379 | 268 | 268 |
| DN4 | 114 | 392 | 400 | 222 | 240 | 375 | 399 | 268 | 272 |
| DN5 | 114 | 396 | 400 | 232 | 240 | 367 | 371 | 268 | 272 |
| DN6 | 110 | 388 | 392 | 224 | 238 | 371 | 371 | 264 | 268 |
| DN7 | 106 | 388 | 404 | 220 | 232 | 371 | 383 | 268 | 276 |
| DN8 | 114 | 392 | 404 | 224 | 234 | 375 | 379 | 264 | 268 |
| DN9 | 102 | 388 | 396 | 230 | 232 | 387 | 395 | 268 | 280 |
| DN10 | 106 | 0 | 0 | 224 | 240 | 367 | 383 | 272 | 300 |
| DN11 | 110 | 392 | 404 | 236 | 236 | 361 | 383 | 268 | 268 |
| DN12 | 114 | 384 | 396 | 224 | 240 | 375 | 379 | 268 | 282 |
| DN13 | 106 | 0 | 0 | 236 | 236 | 371 | 379 | 268 | 272 |
| DN14 | 106 | 392 | 392 | 228 | 228 | 363 | 363 | 268 | 272 |
| DN15 | 102 | 388 | 400 | 240 | 246 | 371 | 379 | 268 | 296 |
| DN16 | 114 | 402 | 404 | 236 | 236 | 379 | 379 | 268 | 276 |
| DN17 | 110 | 388 | 396 | 232 | 232 | 363 | 395 | 272 | 282 |
| DN18 | 102 | 400 | 400 | 228 | 228 | 379 | 379 | 268 | 268 |
| DN19 | 102 | 396 | 404 | 224 | 246 | 375 | 383 | 264 | 280 |
| DN20 | 110 | 392 | 400 | 228 | 228 | 371 | 379 | 264 | 282 |
| DN21 | 110 | 396 | 400 | 236 | 236 | 361 | 387 | 272 | 272 |
| DN22 | 110 | 392 | 396 | 234 | 240 | 379 | 391 | 272 | 282 |
| DN23 | 106 | 392 | 400 | 236 | 236 | 367 | 379 | 268 | 268 |
| DN24 | 102 | 396 | 400 | 236 | 236 | 371 | 375 | 268 | 268 |
| DN25 | 110 | 380 | 382 | 224 | 236 | 371 | 383 | 264 | 268 |
| DN26 | 102 | 396 | 408 | 236 | 236 | 375 | 379 | 260 | 272 |
| DN27 | 102 | 396 | 400 | 230 | 240 | 367 | 367 | 282 | 292 |
| DN28 | 110 | 396 | 400 | 224 | 232 | 379 | 379 | 268 | 268 |
| DN29 | 106 | 400 | 404 | 228 | 232 | 367 | 375 | 264 | 268 |
| DN30 | 110 | 392 | 400 | 228 | 232 | 379 | 383 | 272 | 276 |
| DN31 | 110 | 404 | 408 | 226 | 232 | 375 | 379 | 268 | 280 |
| DN32 | 114 | 0 | 0 | 236 | 240 | 375 | 379 | 256 | 264 |
| DN33 | 110 | 388 | 408 | 226 | 232 | 379 | 383 | 268 | 280 |
| DN34 | 102 | 386 | 396 | 226 | 236 | 363 | 375 | 268 | 272 |
| DN35 | 110 | 400 | 404 | 226 | 236 | 371 | 383 | 268 | 268 |
| DN36 | 0 | 408 | 408 | 232 | 232 | 379 | 379 | 272 | 282 |
| DN37 | 106 | 386 | 408 | 224 | 236 | 371 | 391 | 256 | 268 |
| DN38 | 102 | 396 | 396 | 222 | 232 | 365 | 391 | 272 | 272 |
| DN39 | 110 | 388 | 400 | 228 | 236 | 371 | 371 | 268 | 282 |
| DN40 | 106 | 400 | 404 | 222 | 236 | 367 | 367 | 256 | 268 |
| DN41 | 106 | 396 | 408 | 222 | 236 | 383 | 383 | 264 | 272 |
| DN42 | 106 | 392 | 400 | 236 | 236 | 363 | 375 | 268 | 280 |
| DN43 | 110 | 400 | 404 | 228 | 236 | 379 | 379 | 268 | 296 |
| DN44 | 102 | 400 | 400 | 224 | 224 | 379 | 379 | 276 | 276 |
| DN45 | 110 | 408 | 412 | 224 | 230 | 371 | 371 | 0 | 0 |
| DN46 | 102 | 388 | 400 | 236 | 246 | 367 | 379 | 268 | 282 |
| DN47 | 110 | 396 | 400 | 232 | 236 | 375 | 379 | 268 | 304 |
| DN48 | 106 | 0 | 0 | 222 | 222 | 0 | 0 | 272 | 276 |
| DN49 | 102 | 388 | 404 | 230 | 232 | 365 | 367 | 264 | 264 |
| DN50 | 110 | 388 | 392 | 226 | 236 | 375 | 375 | 268 | 300 |
| DN51 | 110 | 388 | 400 | 224 | 230 | 367 | 383 | 264 | 276 |
| DN52 | 110 | 392 | 396 | 230 | 236 | 365 | 379 | 268 | 282 |
| DN53 | 110 | 0 | 0 | 232 | 236 | 0 | 0 | 272 | 276 |
| DN54 | 110 | 400 | 400 | 236 | 240 | 371 | 371 | 280 | 286 |
| DN55 | 106 | 400 | 400 | 224 | 224 | 379 | 383 | 276 | 276 |
| DN56 | 102 | 396 | 400 | 0 | 0 | 367 | 371 | 268 | 268 |
| DN57 | 110 | 396 | 400 | 230 | 230 | 0 | 0 | 268 | 268 |

Genetic Profiles of Dogs in Community (Part II)

| | PEZ16 | PEZ16 | PEZ17 | PEZ17 | PEZ20 | PEZ20 | PEZ21 | PEZ21 |
|---|---|---|---|---|---|---|---|---|
| DN1 | 292 | 300 | 195 | 195 | 173 | 173 | 85 | 97 |
| DN2 | 304 | 312 | 183 | 199 | 169 | 173 | 85 | 89 |
| DN3 | 292 | 292 | 187 | 191 | 173 | 177 | 97 | 101 |
| DN4 | 284 | 304 | 191 | 195 | 173 | 177 | 85 | 97 |
| DN5 | 300 | 300 | 179 | 199 | 173 | 177 | 97 | 97 |
| DN6 | 288 | 304 | 195 | 199 | 173 | 173 | 85 | 85 |
| DN7 | 300 | 300 | 183 | 195 | 173 | 181 | 89 | 101 |
| DN8 | 292 | 304 | 183 | 187 | 169 | 173 | 93 | 97 |
| DN9 | 300 | 300 | 191 | 199 | 173 | 177 | 93 | 97 |
| DN10 | 292 | 300 | 187 | 191 | 173 | 173 | 89 | 97 |
| DN11 | 300 | 300 | 179 | 187 | 173 | 181 | 93 | 97 |
| DN12 | 292 | 300 | 187 | 199 | 173 | 173 | 93 | 97 |
| DN13 | 292 | 300 | 183 | 187 | 173 | 185 | 85 | 97 |
| DN14 | 292 | 304 | 187 | 191 | 169 | 173 | 101 | 101 |
| DN15 | 284 | 292 | 183 | 187 | 169 | 181 | 97 | 97 |
| DN16 | 288 | 300 | 187 | 187 | 173 | 177 | 89 | 89 |
| DN17 | 300 | 300 | 191 | 195 | 169 | 173 | 89 | 97 |
| DN18 | 296 | 300 | 191 | 199 | 173 | 173 | 85 | 85 |
| DN19 | 292 | 300 | 183 | 187 | 181 | 181 | 89 | 93 |
| DN20 | 296 | 304 | 191 | 195 | 173 | 181 | 97 | 97 |
| DN21 | 292 | 292 | 183 | 191 | 173 | 177 | 93 | 97 |
| DN22 | 300 | 300 | 187 | 191 | 173 | 173 | 89 | 93 |
| DN23 | 296 | 300 | 183 | 191 | 173 | 173 | 93 | 97 |
| DN24 | 288 | 300 | 195 | 203 | 173 | 173 | 89 | 97 |
| DN25 | 300 | 304 | 187 | 195 | 173 | 173 | 85 | 97 |
| DN26 | 296 | 300 | 183 | 187 | 177 | 177 | 89 | 93 |
| DN27 | 300 | 300 | 183 | 191 | 173 | 177 | 89 | 97 |
| DN28 | 300 | 308 | 191 | 199 | 181 | 181 | 89 | 89 |
| DN29 | 300 | 300 | 183 | 183 | 173 | 177 | 93 | 101 |
| DN30 | 296 | 296 | 195 | 195 | 169 | 169 | 89 | 89 |
| DN31 | 296 | 300 | 191 | 195 | 173 | 173 | 85 | 89 |
| DN32 | 300 | 300 | 187 | 195 | 169 | 177 | 85 | 101 |
| DN33 | 300 | 334 | 195 | 195 | 173 | 173 | 85 | 89 |
| DN34 | 296 | 304 | 187 | 191 | 173 | 181 | 93 | 97 |
| DN35 | 288 | 296 | 187 | 191 | 177 | 177 | 97 | 101 |
| DN36 | 288 | 316 | 195 | 203 | 0 | 0 | 97 | 97 |
| DN37 | 284 | 288 | 183 | 183 | 0 | 0 | 97 | 101 |
| DN38 | 292 | 300 | 191 | 199 | 173 | 173 | 97 | 97 |
| DN39 | 292 | 308 | 187 | 187 | 169 | 181 | 85 | 85 |
| DN40 | 292 | 316 | 187 | 195 | 173 | 177 | 85 | 85 |
| DN41 | 300 | 300 | 187 | 191 | 173 | 173 | 97 | 97 |
| DN42 | 296 | 300 | 191 | 195 | 173 | 173 | 89 | 101 |
| DN43 | 292 | 300 | 195 | 195 | 173 | 173 | 89 | 93 |
| DN44 | 300 | 300 | 187 | 191 | 169 | 173 | 89 | 89 |
| DN45 | 296 | 300 | 191 | 195 | 169 | 173 | 89 | 93 |
| DN46 | 284 | 296 | 183 | 195 | 169 | 181 | 97 | 97 |
| DN47 | 308 | 316 | 187 | 195 | 169 | 173 | 97 | 97 |
| DN48 | 292 | 300 | 187 | 191 | 169 | 173 | 97 | 97 |
| DN49 | 292 | 304 | 183 | 183 | 169 | 169 | 93 | 97 |
| DN50 | 300 | 316 | 187 | 195 | 169 | 181 | 97 | 97 |
| DN51 | 308 | 316 | 183 | 191 | 173 | 181 | 89 | 89 |
| DN52 | 308 | 308 | 179 | 183 | 177 | 181 | 97 | 101 |
| DN53 | 296 | 316 | 191 | 199 | 173 | 177 | 97 | 101 |
| DN54 | 292 | 296 | 183 | 191 | 173 | 181 | 89 | 101 |
| DN55 | 300 | 304 | 187 | 187 | 169 | 173 | 89 | 89 |
| DN56 | 288 | 296 | 187 | 195 | 169 | 173 | 89 | 89 |
| DN57 | 296 | 300 | 179 | 183 | 181 | 181 | 89 | 89 |

TABLE 2
Genetic Profile of TD0002113

| | | Allele 1 | Allele 2 |
|---|---|---|---|
| TD0002113 | Amelogenin | 0 | 0 |
| TD0002113 | FH2010 | 0 | 0 |
| TD0002113 | FH2054 | 0 | 0 |
| TD0002113 | FH2079 | 271 | 271 |
| TD0002113 | FH2361 | 0 | 0 |
| TD0002113 | PEZ01 | 118 | 122 |
| TD0002113 | PEZ03 | 108 | 124 |
| TD0002113 | PEZ05 | 102 | 110 |
| TD0002113 | PEZ06 | 396 | 404 |
| TD0002113 | PEZ08 | 0 | 0 |
| TD0002113 | PEZ11 | 363 | 379 |
| TD0002113 | PEZ12 | 264 | 264 |
| TD0002113 | PEZ16 | 292 | 300 |
| TD0002113 | PEZ17 | 195 | 195 |
| TD0002113 | PEZ20 | 173 | 173 |
| TD0002113 | PEZ21 | 85 | 97 |

The genotype for TD0002113 was then compared against the community from which the sample was collected. This community contained genotypes for 57 unique canines, as discussed above. Comparison of TD0002113 against this community identified DN1 as the DNA match.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description and shown in the accompanying drawings shall be interpreted as illustrative and not in a limiting sense.

What is claimed is:

1. A method for determining the source of animal waste, the method comprising:
collecting a DNA sample of animals residing in the community;
conducting DNA analysis on the DNA samples to develop a genetic profile associated with each sample;
preparing a database of the genetic profiles;
collecting a sample of waste from an unknown animal;
conducting DNA analysis of the sample of waste to develop a genetic profile of the animal from which the sample of waste originated; and
comparing the genetic profile from the sample of waste to the genetic profiles in the database.

2. The method according to claim 1, wherein the database is stored on a computer-readable medium.

3. The method according to claim 1, wherein the step of comparing the genetic profiles is conducted manually.

4. The method according to claim 1, wherein the genetic profile is prepared using one or more of hybridization, Polymerase Chain Reaction, size fractionation, DNA sequencing, DNA microarrays, high density fiber-optic arrays of beads, primer extension, mass spectrometry, and whole-genome sampling.

5. The method according to claim 1, further comprising extracting DNA from the sample of waste before conducting the DNA analysis step.

6. The method according to claim 1, further comprising stabilizing the sample of waste with a DNA stabilizer.

7. The method according to claim 1, wherein the database contains between about 1 and 1000 genetic profiles.

8. The method according to claim 1, wherein the database contains between about 1 and 500 genetic profiles.

9. A computer readable medium comprising:
a. a database having stored thereon genetic profiles developed from DNA analysis of a set of known animals and
b. computer-executable instructions for implementing a method for comparing a genetic profile from an unknown animal with the genetic profiles stored on the database for determining the source of the unknown genetic profile.

10. A kit for collecting and analyzing animal waste, the kit comprising:
a. at least one first DNA sample collector for collecting DNA samples from a known group of animals; and
b. at least one second DNA sample collector for collecting a fecal sample from an unknown animal.

11. The kit according to claim 10, wherein the at least one DNA sample collector is designed to collect one or more of cheek cells, saliva, fur, blood, or fecal matter.

12. The kit according to claim 10, comprising a plurality of the first DNA sample collector.

13. The kit according to claim 10, wherein the at least one second DNA sample collector further includes a DNA stabilizer.

14. The kit according to claim 10, further comprising a DNA stabilizer.

15. The kit according to claim 10, comprising a plurality of the second DNA sample collector.

16. The kit according to claim 10, further including a buccal swab.

17. The kit according to claim 10, further including a syringe and a needle.

18. The kit according to claim 10, further comprising a scoop.
